(19) United States
(12) Patent Application Publication
(10) Pub. No.: US 2004/0006318 A1
(43) Pub. Date: Jan. 8, 2004

(54) Side hole in catheter

(76) Inventors: Ramanathan Periakaruppan, Plymouth, MN (US); Scott D. Moore, Columbia Heights, MN (US)

Correspondence Address: MARSHALL, GERSTEIN & BORUN, Michael R. Hull, 233 S. Wacker Drive, Suite 6300, Sears Tower, Chicago, IL, 60606-6357, US

(21) Appl. No.: 10/187,922

(22) Filed: Jul. 2, 2002

(51) Int. Cl. A61M 5/00

(52) U.S. Cl. 604/264; 264/632; 264/145; 604/154

(57) ABSTRACT

A catheter includes an elongated tubular member having a lumen extending through the elongated tubular member. The elongated tubular member also has an outer surface, a distal end and at least one opening. The opening extends between the inner and outer surfaces, provides communication between the outer surface and the lumen and includes at least two straight edges extending parallel to the lumen of the tubular member.

### TECHNICAL FIELD

Catheters are disclosed which are used to access and treat target areas in the vascular system and which have collapsible-resistant side holes for communication of fluid between the vascular system and catheter and vice versa.

### BACKGROUND

Guide catheters and diagnostic catheters are well known for use in coronary catheterization and percutaneous transluminal coronary angioplasty (PTCA) procedures. Guide catheters aid in treatment of arterial lesions by providing a conduit for positioning dilation balloon systems across an arterial stenosis. Guide catheters and diagnostic catheters work with various assemblies for performing other medical, therapeutic, and diagnostic procedures, such as dye delivery, arterial flushing, or arterial pressure monitoring.

Diagnostic catheters are used during cardiac catheterization for diagnosis of coronary artery disease in order to define vessel anatomy, isolate lesions, and identify adjacent cardiac branches which may impinge on the lesion and affect ventricular function.

For treatment of the coronary disease through angioplasty or other catheter based treatments, guide catheters are used. Guide catheters provide access to the area within the arterial system containing the stenotic lesion and support for the treatment catheter. Guide catheters typically have a pre-shaped distal section or tip region to aid in accessing the ostium of the coronary artery to receive treatment. Typically, this distal pre-shaped section is curved such that the distal end resembles a crook.

Catheters are often curved to provide support against the aortic wall when seated within the ostium and to resist the tendency for a catheter to “pop out” of the ostium (termed “backout force”) when injecting dye or advancing a treatment catheter farther into the artery. During insertion of the catheter and during use of the catheter, the curved distal portions of the catheter usually become lodged in the ostium. To reduce the risk of blood flow through the ostium being impeded by the catheter, many catheters include side holes on the inner side of the distal curved portion to allow blood to flow between the lumen of the catheter and surrounding tissues. This communication through the inner side holes helps to compensate for the loss of blood circulation through the ostium which is at least partially obstructed by the catheter.

Many current catheters include side holes on the inner side of the curve because physicians have expressed a preference for holes on the inner side of the curve. Further, it is advantageous to have side holes in the inner side of the curved portion of the distal end of the catheter to allow blood to continually perfuse the myocardium while the catheter is at least partially blocking the ostium. These side holes can maintain an adequate blood flow thereby preventing necrosis of the myocardial tissue due to a lack of oxygen.

Presently, catheters are specifically manufactured with high curve retention to maintain catheter placement within the ostium and to resist backout forces. Additionally, to minimize unwanted kinking or bending of the catheter during placement in the artery or during use, some catheters are manufactured that include an inner layer commonly formed of polytetrafluoroethylene, a middle layer consisting of braided wire for torque control, and a third, outer layer commonly formed of polyethylene, polyurethane or a nylon-blend. These three layers provide for stable positioning of the catheter and backout support during treatment procedures. The braid of high-strength fibers or stainless steel wires located between the liner and the outer covering helps to make the catheters kink resistant.

Although catheters are often designed to eliminate undesirable “winking” or collapsing of the side holes during manufacture and use, many side holes still have a tendency to wink or collapse during formation of the curve or during use. In many current manufacturing processes, circular side holes are punched into the distal end of the catheter before curving the distal end. Then, as the distal end is curved, the holes may become distorted which can reduce the flow of blood through the side holes. Occasionally, the circular side hole collapses during curving thereby impeding the blood flow through the side holes.

At present, the most common shape for the side hole is a round or circular shape. The surface tension forces created during curving of the catheter are concentrated at two points on the round side hole which fall along an axis or diameter of the hole perpendicular to the longitudinal axis of the catheter. Surface tension forces which are generated during the curving portion of the manufacturing process can cause the round holes to collapse or partially close, which is undesirable.

Therefore there is a need for an improved side hole design in the distal curved section of certain catheters which avoids closing, collapsing or winking of the holes during manufacture and subsequent use.

### SUMMARY OF THE DISCLOSURE

An elongated tubular member is disclosed which comprises a lumen extending through the elongated tubular member, an outer surface, and a distal end. The tubular member also has at least one opening extending between the inner and outer surfaces for providing communication between the outer surface and the lumen. The opening includes at least two straight or substantially straight edges which extend parallel to the lumen of the tubular member. The distal end of the catheter is curved and includes an exterior curved surface, an interior curved surface and two side surfaces. The opening is preferably located in the curved surface of the distal end and, more specifically, in the interior curved surface.

A method for manufacturing a catheter with distal side holes is disclosed which includes creating a straight elongated tubular member having a distal end and a proximal end, punching into the distal end of the tubular member at least one opening, the at least one opening having two straight edges extending parallel to a lumen of the tubular member and two rounded edges, and curving the distal end of the tubular member to create an interior curved surface and an exterior curved surface with the opening disposed in the interior curved surface.

### BRIEF DESCRIPTION OF THE DRAWINGS

FIG. 1 is a partial perspective view illustrating a distal end of a disclosed catheter having a curved elongated tubular member and two side holes;

FIG. 2 is a partial plan view of the elongated tubular member and side holes of FIG. 1;

FIG. 3 is a sectional view of the elongated member of FIG. 2 taken along line 3-3 of FIG. 2;

FIG. 4 is a plan view of an alternative side hole design;

FIG. 5 is a plan view of another alternative side hole design;

FIG. 6 is a plan view of another alternative side hole design;

FIG. 7 is a plan view of another alternative side hole design;

FIG. 8 is a plan view of another alternative side hole design;

FIG. 9 is a plan view of another alternative side hole design;

FIG. 10 is a plan view of another alternative side hole design;

FIG. 11 is a plan view of another alternative side hole design; and

FIG. 12 is a plan view of another alternative side hole design.

### DETAILED DESCRIPTION OF THE PRESENTLY PREFERRED EMBODIMENTS

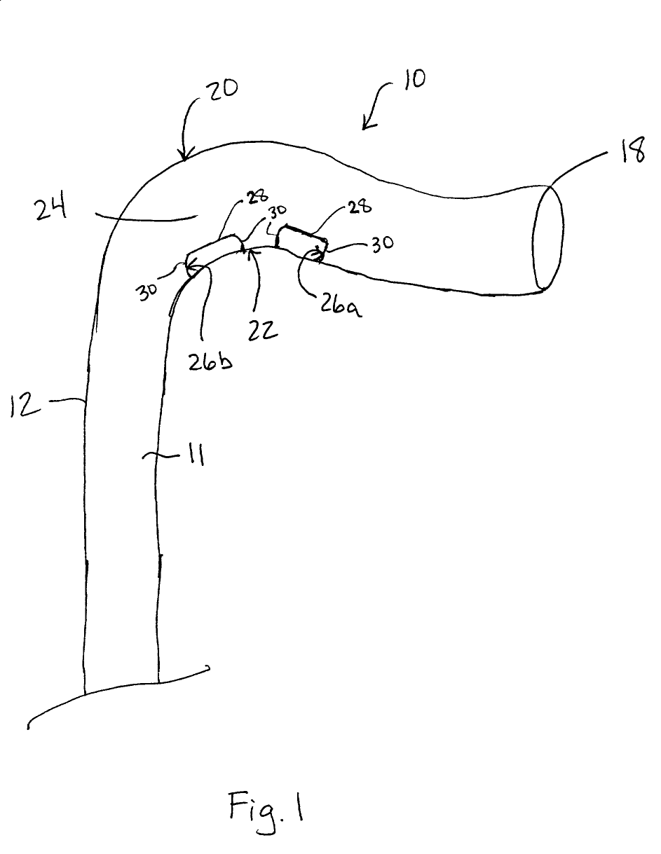

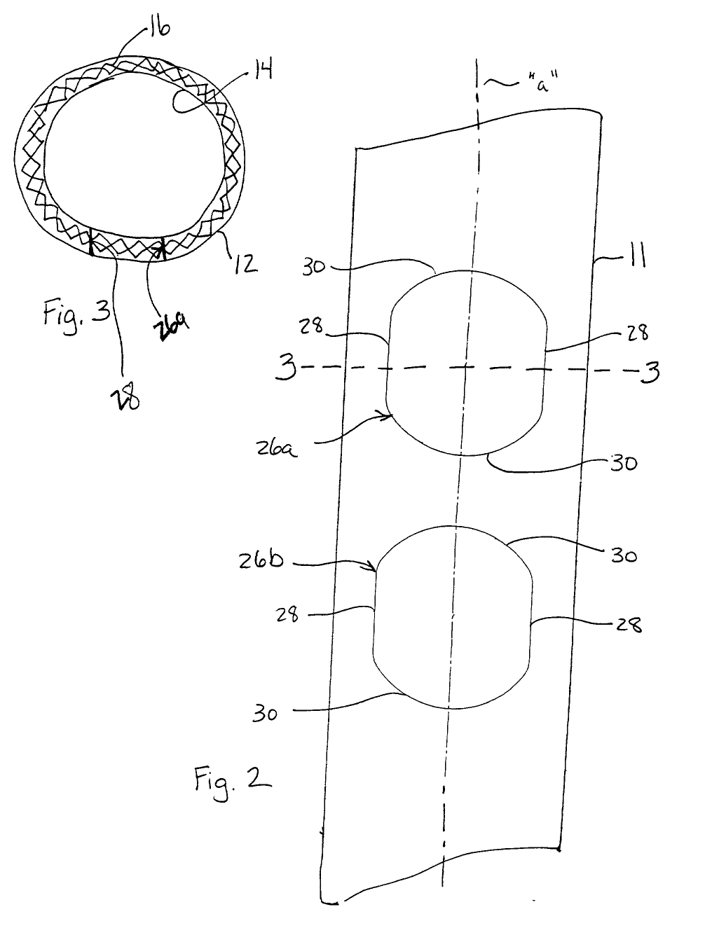

Referring now to the drawings, and with specific reference to FIGS. 1-3, a catheter 10 having an elongated tubular member 11 is shown. The elongated tubular member 11 includes an outer surface 12 and an inner surface 14 that defines a lumen. The outer surface 12 is commonly formed of polyethylene, polyurethane, a nylon blend or any other polymer possessing similar properties and known to those skilled in the art. The inner surface 14 is commonly formed of polytetraflouroethylene. A middle layer 16 of braided wire is shown between the outer surface 12 and the inner surface 14. The middle layer 16 which is a braid of high-strength fibers or stainless steel wires as is known by those skilled in the art helps to provide the catheter with strength and resistance to kinking or bending during use of the catheter. While the braided wire middle layer 16 may be found in a preferred embodiment, the catheter may also be manufactured without the braided wire layer.

FIG. 1 shows a distal end 18 of the elongated tubular member 11. The distal end is curved to provide support against the aortic wall when the catheter is seated with the ostium. Additionally, this curved shape facilitates placement of the catheter and helps resist the tendency for a catheter to become dislodged from or “pop out” of the ostium during use. The outer surface 12 of the curved distal end 18 includes an exterior curved surface 20, an interior curved surface 22 and two side curved surfaces 24. In a preferred embodiment of the invention, two side holes 26a, 26b are located in the curved distal end 18 of the elongated tubular member 11. As illustrated in FIG. 1, the side holes 26a, 26b are preferably located in the interior curved surface 22 of the distal end 18. It is advantageous to have these holes 26a, 26b in the interior curved surface 22 to allow blood to perfuse the myocardium. If blood flow through the ostium is temporarily reduced during insertion or placement of the catheter in the ostium, these holes help to maintain a supply of blood to the myocardium thereby decreasing the risk of ischemia or oxygen loss to the myocardium.

As can be seen in FIG. 2, each of the side holes 26a, 26b has an elongate shape comprising of two straight edges 28 and two rounded ends 30. The straight edges are parallel to the imaginary longitudinal axis extending throughout the elongated tubular member 11, parallel to the lumen of the elongated tubular member and represented by dashed line “a.” The two straight edges 28 are improvement over the current circular side holes which are used most commonly in guide catheters. The straight edges 28 better distribute the surface tension forces created during the manufacturing process of catheters having curved distal ends.

For example, circular side holes have two stress points (not shown), one on either side of the hole and along an imaginary radial line that runs perpendicular to the imaginary longitudinal axis “a” of the elongated tubular member 11 where the stress forces become concentrated. As the catheters are curved during manufacture, these two concentrated stress points are frequently the spot of collapse or buckling of the side hole.

With openings 26a, 26b having two straight edges 28 and two rounded ends 30, the stresses created by surface tension are distributed over a larger portion of each side hole circumference. This distribution of stresses reduces the distortion to the hole during the curving process and reduces the likelihood of hole collapse. The straight edges 28 create a more equal distribution of stress on numerous points along each edge 28. The shape of the side holes 26a, 26b is designed to have the same open area as the conventional round holes. This will allow the same blood flow through the side holes 26a, 26b without increasing contrast loss through the side holes.

Further, the elongated side holes 26a, 26b are desirable because a surgeon utilizing a catheter having elongated holes may more easily cover the holes with his/her thumb to close the holes and reduce blood leakage during a surgical procedure. For example, the elongated shape of the holes 26a, 26b is easier to control because the surgeon may exert less pressure with his/her thumb to control the blood pressure in the catheter. Specifically, it is common for a surgeon to cover catheter side holes by placing his/her thumb on the outer surface of the catheter parallel to the longitudinal axis of the catheter. Because of the elongated shape of the holes 26a, 26b, the holes 26a, 26b have a greater amount of exposed area along the longitudinal axis of the catheter and, similarly, along the lengthwise axis of the surgeon's thumb. For this reason, a large percentage of each opening 26a, 26b falls under the middle part of the surgeon's thumb which allows him/her to exert less pressure to keep his thumb over the opening 26a, 26b and control leakage. In contrast, round holes have a greater percentage of exposed area that exists farther away from the longitudinal axis of the catheter. The greater width of the round holes requires a surgeon to exert more pressure with his/her thumb to keep the openings covered to control leakage. Thus, it is more difficult and more tiresome for the surgeon to control blood leakage from round-shaped catheter side holes.

As shown in FIG. 2, the elongated side holes 26a, 26b have rounded or radiused ends 30. While most round-shaped side holes have a uniform radius of about 0.028 inch, the elongated holes have various lengths. For example, one embodiment of the elongated side hole has a length between curved ends 30, parallel to the longitudinal axis “a” of the catheter, which is approximately 0.030 inch and a width between the two straight edges 28 which is approximately 0.025 inch. The distance between the centers of the two adjacent side holes 26a, 26b is approximately 0.150 inch. The distance from the center of the most distal side hole 26a to the distal end 18 of the elongated tubular member 11 is preferably about one inch.

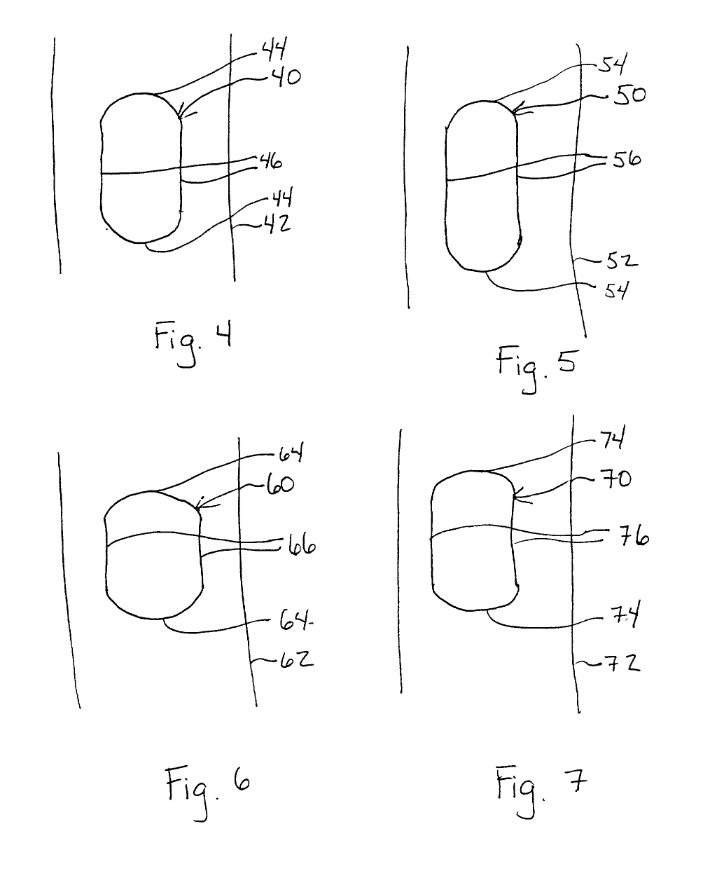

Now, referring to FIGS. 4-12, while the elongated side holes 26a, 26b may have a variety of different shapes, each embodiment does have straight edges extending about the center point of the hole and parallel to the longitudinal axis of the catheter for distribution of the stresses on the hole during the curving process. The side holes of FIGS. 4-12 are merely illustrative of some alternative embodiments of the side hole shape and should not be limited to those which are described herein. Additionally, even though one side hole is depicted in each of the FIGS. 4-12, an alternative embodiment utilizing any of the hereinafter described side holes may have two holes or as many holes as required to accomplish a desired purpose which is commonly perfusion of the myocardium. As described previously in connection with side holes 26a, 26b, the preferred distance between the centers of the two adjacent side holes described hereinafter is approximately 0.15 inch. Similarly, the preferred distance from the center of the most distal side hole hereinafter described to the distal end of the elongated tubular member is preferably about one inch.

As is shown in FIG. 4, a side hole 40 of a catheter 42 may a length between curved ends 44, parallel to the longitudinal axis of the catheter 42, which is approximately 0.035 inch and a width between the two straight edges 46 which is approximately 0.020 inch.

As is shown in FIG. 5, a side hole 50 of a catheter 52 may a length between curved ends 54, parallel to the longitudinal axis of the catheter 52, which is approximately 0.040 inch and a width between the two straight edges 56 which is approximately 0.017 inch.

As is shown in FIG. 6, a side hole 60 of a catheter 62 may a length between curved ends 64, parallel to the longitudinal axis of the catheter 62, which is approximately 0.030 inch and a width between the two straight edges 66 which is approximately 0.023 inch.

As is shown in FIG. 7, a side hole 70 of a catheter 72 may a length between curved ends 74, parallel to the longitudinal axis of the catheter 72, which is approximately 0.033 inch and a width between the two straight edges 76 which is approximately 0.020 inch.

As is shown in FIG. 8, a side hole 80 of a catheter 82 may a length between curved ends 84, parallel to the longitudinal axis of the catheter 82, which is approximately 0.040 inch and a width between the two straight edges 86 which is approximately 0.016 inch.

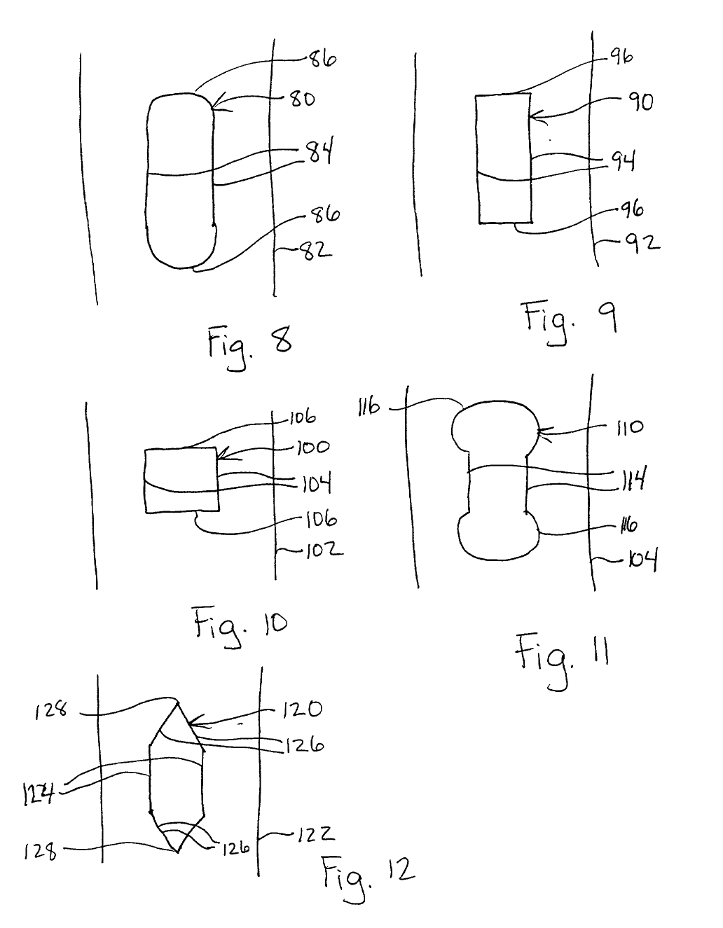

FIG. 9 shows an additional alternate embodiment of a side hole 90 of a catheter 92. Side hole 90 has two straight edges 94 extending parallel to the longitudinal axis of the catheter 92 and two straight-edged ends 96 joining the edges 94 to form a rectangular-shaped hole 90. Further, FIG. 10 shows an alternate embodiment of a side hole 100 of a catheter 102. Side hole 100 has two straight edges 104 extending parallel to the longitudinal axis of the catheter 102 and two straight-edged ends 106, which are the same length as edges 104, and join edges 104 to form a square-shaped hole 100.

As illustrated in FIG. 11, an alternate embodiment of a side hole 110 of a catheter 112 may have a bone-like shape. Side hole 110 has two straight edges 114 extending parallel to the longitudinal axis of the catheter 112 and two bulbous ends 116 joining the straight edges 114 to form the bone-like shaped hole 110.

Yet another alternate embodiment of a side hole 120 of a catheter 122 is illustrated in FIG. 12. Side hole 120 has two straight edges 124 extending parallel to the longitudinal axis of the catheter 112 and two straight edges 126 which extend from edges 124 and come together to form a point 128 at both ends of the side hole 20.

Although the side holes are illustrated in FIG. 1 in the curved portion of the distal end 18 of the elongated tubular member 11, the elongated side holes 26a, 26b may be utilized advantageously in alternative embodiments of any curved portion of an elongated tubular member of a catheter. Additionally, two side holes are generally depicted (see FIGS. 1-2), an alternative design could have only one side hole or could have three or more side holes to facilitate perfusion of the myocardium and other targeted tissues.

The disclosed designs have been described in terms of several exemplary embodiments, one of ordinary skill in the art will appreciate that the disclosed concepts may be otherwise embodied without departing from the scope and spirit of the disclosure as set forth in the appended claims.

What is claimed is:

1. A catheter comprising:
an elongated tubular member having a lumen extending through the elongated tubular member, the elongated tubular member further comprising an outer surface, a distal end, and at least one opening providing communication between the outer surface and the lumen and having at least two substantially straight edges extending parallel to the lumen.

2. The catheter of claim 1 wherein the distal end includes a curved portion and the at least one opening is disposed in the curved portion.

3. The catheter of claim 2 wherein the curved portion has an exterior curved surface, an interior curved surface and two side curved surfaces and the opening is disposed in the interior curved surface.

4. The catheter of claim 1 wherein the at least one opening further comprises two rounded ends.

5. The catheter of claim 1 wherein the at least one opening further comprises two substantially straight ends.

6. The catheter of claim 1 wherein the at least one opening further comprises two pointed ends.

7. A catheter comprising:
an elongated tubular member having a lumen extending through the elongated tubular member, the elongated tubular member further comprising an outer surface, a distal end, a curved portion and an opening means in the curved portion providing communication between the outer surface and the lumen.

8. The catheter of claim 7 wherein the curved portion has an exterior curved surface, an interior curved surface and two side curved surfaces and the opening means is disposed in the interior curved surface.

9. The catheter of claim 7 wherein the opening means comprises two straight edges extending parallel to the lumen of the tubular member.

10. The catheter of claim 9 wherein the opening means further comprises two rounded ends.

11. The catheter of claim 9 wherein the opening means further comprises two substantially straight ends.

12. A method for manufacturing a catheter with at least one distal side opening comprising the steps of:
creating a straight elongated tubular member having a distal end and a proximal end;
punching into the distal end of the tubular member at least one opening having two straight edges extending parallel to a lumen of the tubular member; and
curving the distal end of the tubular member to create an interior curved surface and an exterior curved surface and to position the opening in the interior curved surface.

13. The method of manufacturing of claim 12 wherein the at least one opening further includes two round ends.

14. The method of manufacturing of claim 12 wherein the at least one opening further includes two substantially straight ends.

15. The method of manufacturing of claim 12 wherein the at least one opening further includes two pointed ends.
